(19) United States
(12) Patent Application Publication
(10) Pub. No.: US 2026/0151108 A1
(43) Pub. Date: Jun. 4, 2026

(54) ULTRASOUND PROBE METAL ENCLOSURE THERMAL ISOLATION

(71) Applicant: GE Precision Healthcare LLC, Waukesha, WI (US)

(72) Inventors: Yuji Okumura, Hino (JP); Mayuki Nakamura, Hino (JP)

(21) Appl. No.: 19/397,524

(22) Filed: Nov. 21, 2025

(30) Foreign Application Priority Data
Nov. 29, 2024 (JP) 2024-208057

(51) Int. Cl. A61B 8/00

(52) U.S. Cl. CPC A61B 8/4444

(57) ABSTRACT

An ultrasonic irradiating device including an ultrasonic vibrator disposed in front of the ultrasonic irradiating device; an outer case surrounding the ultrasonic vibrator; and a heat-conduction component thermally connected to the ultrasonic vibrator and transferring heat generated by the ultrasonic vibrator. The outer case includes a front portion made of metal, a rear portion, and a heat-insulation component disposed between the front portion and the rear portion, and the rear portion of the outer case and the heat-conduction component are thermally connected.

### CROSS-REFERENCE TO RELATED APPLICATION

This application claim priority to Japanese Patent Application No. 2024-208057, which was file on November 29, 2024 at the Japanese Patent Office. The entire contents of the above-listed application are incorporated by reference herein in their entirety.

### TECHINICAL FIELD

The present invention relates to an ultrasonic irradiating device, and more particularly to a heat-insulation component for an outer case of an ultrasonic irradiating device.

### BACKGROUND

Ultrasonic irradiating devices have been used as: ultrasound diagnostic devices for acquiring ultrasound images of living organisms and non-living structures; ultrasonic treatment devices for destroying tumors and stones in living organisms; nerve stimulating devices for stimulating nerves in vivo to release prescribed neurotransmitters; and the like.

Such an ultrasonic irradiating device may be exposed to physical impacts or high external stresses. Physical impacts or high external stresses on an ultrasonic irradiating device may be caused by the device being dropped onto a floor or by an erroneous operation or action by an operator or transporter. Physical impacts or high external stresses on an ultrasonic irradiating device can damage a resin outer case that conventional ultrasonic irradiating devices generally have. Therefore, there is a need for an outer case that will not be damaged even if the ultrasonic irradiating device is subjected to physical impact or high external stress. Furthermore, for example, in the case of an ultrasound image diagnostic device or the like used by military doctors on the battlefield, the outer case of the ultrasonic irradiating device may be required to have high durability and high impact resistance depending on the expected environment of use.

Metal materials often have higher durability and impact resistance than the resin materials that have been used as outer cases for conventional ultrasonic irradiating devices. Therefore, it may be possible to consider forming an outer case of an ultrasonic irradiating device from metal.

However, if a tip-end part of an outer case of an ultrasonic irradiating device is formed from a metal, the heat capacity of the tip-end part of the outer case increases, making it impossible to use an ultrasonic irradiating device with a metal outer case in various temperature environments. In particular, in applications where a tip-end part of an outer case of an ultrasonic irradiating device is required to come into contact with a living organism such as a human or non-human animal, there is a problem in that the living organism feels cold when the tip-end part of the outer case of the ultrasonic irradiating device comes into contact with the living organism. It is particularly important to avoid causing stress due to temperature differences when contacting children or experimental animals. Furthermore, if the temperature of the tip-end part of the outer case of the ultrasonic irradiating device is below the freezing point, the tip-end part of the outer case of the ultrasonic irradiating device may stick to the skin of a living organism, preventing immediately separation of the two.

Conversely, there are cases where the temperature of the tip-end part of the outer case of the ultrasonic irradiating device becomes equal to or higher than the temperature of a living organism subject to contact therewith. An ultrasonic vibrator provided in the ultrasonic irradiating device generates ultrasonic waves by vibration an element thereof, and this vibration generates heat. Furthermore, an electronic component included in an outer case of an ultrasonic irradiating device may also be a heat generation source. These heat generation sources are preferably prevented from raising the temperature of a tip-end part of an outer case of an ultrasonic irradiating device to an undesirable level. If the temperature of the tip-end part of the outer case of the ultrasonic irradiating device rises to an undesirable level, the living organism subject to contact with the tip-end part may become stressed.

Furthermore, even when the subject of contact with the ultrasonic irradiating device is a non-living structure, the temperature of the tip-end part of the outer case of the ultrasound irradiation device may cause problems. As is clear to a person of ordinary skill in the art, the speed of ultrasonic waves varies with temperature. If the temperature at a tip-end part of an outer case of an ultrasonic irradiating device causes a non-uniform temperature distribution on the subject of contact, a problem occurs where the speed at which the ultrasonic waves travel inside the subject of contact becomes non-uniform.

### SUMMARY

In a first aspect of the present disclosure, an ultrasonic irradiating device is provided. The ultrasonic irradiating device includes: an ultrasonic vibrator disposed in front of the ultrasonic irradiating device; an outer case surrounding the ultrasonic vibrator; and a heat-conduction component thermally connected to the ultrasonic vibrator and transferring heat generated by the ultrasonic vibrator. The outer case includes (1) a front portion made of metal, (2) a rear portion, and (3) a heat-insulation component disposed between the front portion and the rear portion, and the rear portion of the outer case and the heat-conduction component are thermally connected.

In a second aspect of the present disclosure, an ultrasound diagnostic system is provided. The ultrasound diagnostic system includes: an ultrasonic probe for obtaining an ultrasound image; an ultrasonic irradiating device having the features of the first aspect of the present disclosure; a processor for processing an echo signal received from the ultrasonic probe using an image generation program to generate an ultrasound image; and a non-transitory storing medium for storing the image generation program.

In a third aspect of the present disclosure, a heat-insulation component for an outer case of an ultrasonic irradiating device is provided. The ultrasonic irradiating device includes: an ultrasonic vibrator disposed in front of the ultrasonic irradiating device; an outer case surrounding the ultrasonic vibrator; and a heat-conduction component thermally connected to the ultrasonic vibrator and transferring heat generated by the ultrasonic vibrator. The outer case includes (1) a front portion made of metal, (2) a rear portion, and (3) a heat-insulation component disposed between the front portion and the rear portion, and the rear portion of the outer case and the heat-conduction component are thermally connected.

In a fourth aspect of the present disclosure, a method is provided for manufacturing an ultrasonic irradiating device having the features of the first aspect of the present disclosure. The method for manufacturing an ultrasonic irradiating device includes: a step for fabricating the heat-insulation component by at least one of injection molding, extrusion molding, blow molding, vacuum molding, compression molding, 3D printing, and cutting and processing; a step for connecting the ultrasonic vibrator and a front-end part of the heat-conduction component; a step for securing the ultrasonic vibrator to the front portion of the outer case; a step for joining the heat-insulation component to the front portion of the outer case by an adhesive; a step for thermally connecting the heat-conduction component to the rear portion of the outer case; and a step for joining the heat-insulation component to the rear portion of the outer case by an adhesive.

### BRIEF DESCRIPTION OF THE DRAWINGS

FIG. 1 is a block diagram depicting one example of a schematic configuration of an ultrasound diagnostic system according to an embodiment;

FIG. 2 is a diagram depicting an internal structure of an ultrasonic irradiating device according to an embodiment;

FIG. 3 is a diagram depicting an internal structure of an ultrasonic irradiating device according to an embodiment;

FIG. 4 is an enlarged view of a heat-insulation component according to an embodiment;

FIG. 5 is an enlarged view of another heat-insulation component according to an embodiment;

FIG. 6 is a cross-sectional view of the heat-insulation component of FIG. 5 according to an embodiment;

FIG. 7 is an exploded perspective view depicting an internal structure of an ultrasonic probe according to an embodiment; and

FIG. 8 is a flowchart depicting a method for manufacturing an ultrasonic probe according to an embodiment.

### DETAILED DESCRIPTION

Embodiments of the present invention will be described below. Note that the invention claimed in the embodiments described herein is not limited. In particular, in the present disclosure, a medical ultrasound image diagnostic system is described as an example. However, the present invention may be applied to an ultrasonic examination system, an ultrasonic examination device, and an ultrasonic probe for non-destructive examination of buildings, structures, various mechanical devices, and the like. Furthermore, the present invention can also be embodied as: an ultrasonic treatment device for destroying tumors and stones in vivo; a nerve stimulating device for stimulating a nerve in vivo to release a prescribed neurotransmitter; and the like.

Furthermore, throughout the present specification and the claims, scope limiting components can be combined and interchanged unless the context or language dictates otherwise.

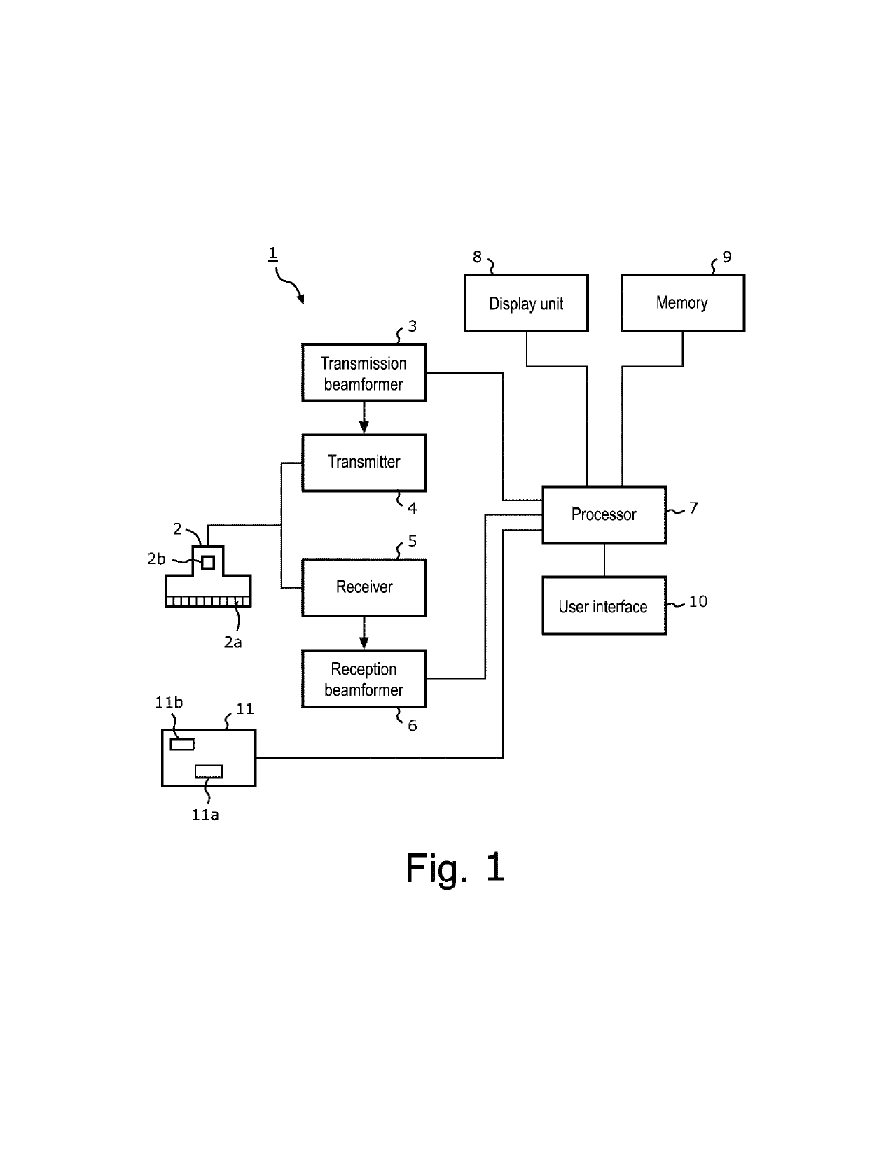

Embodiments of the present invention will be described hereinafter with reference to the drawings. FIG. 1 is a block diagram of an ultrasound diagnostic system 1.

The ultrasound diagnostic system 1 has an ultrasonic probe 2, a transmission beamformer 3, a transmitter 4, a receiver 5, a reception beamformer 6, a processor 7, a display unit 8, a memory 9, and a user interface 10. The ultrasonic probe 2 is an example of an ultrasonic irradiating device of the present invention.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2 via a built-in electronic component 2b, and ultrasonic waves are transmitted from the vibrating elements 2a. The ultrasonic waves transmitted from the vibrating element 2a are reflected inside the subject, and a reflection echo is received by the vibrating element 2a. The vibrating elements 2a convert the received echo to an electrical signal and output this electrical signal as an echo signal to the receiver 5 via the built-in electronic component 2b. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or a plurality of processors, including one or a plurality of: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

If the ultrasonic irradiating device is embodied not as an ultrasound diagnostic device for acquiring an ultrasound image of a living organism and a non-living structure, but as an ultrasonic treatment device for destroying a tumor and stone in vivo, or as a nerve stimulating device for stimulating a nerve in vivo to release a prescribed neurotransmitter, at least the receiver 5 and the reception beamformer 6 become unnecessary components.

Returning again to the example of the ultrasound diagnostic system, the processor 7 controls the transmission beamformer 3, the transmitter 4, the receiver 5, and the reception beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display unit 8. The processor 7 can process echo data to generate an ultrasound image. The term “electronic communication” may be defined to include both wired and wireless communications. The processor 7 may include a central processing unit (CPU) according to one embodiment. According to another embodiment, the processor 7 may include one or more processor, another electronic component that may execute a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

Furthermore, the processor 7 may generate various ultrasound images (e.g., a B-mode image, color Doppler image, M-mode image, color M-mode image, spectral Doppler image, elastography image, TVI image, strain image, strain rate image, and the like) on the basis of data obtained by processing via the reception beamformer 6. In addition, one or a plurality of modules can generate these ultrasound images.

An image beam and/or an image frame may be saved and timing information may be recorded indicating when the data is retrieved to the memory. The module may include, for example, a scan conversion module that performs a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasound images may be read from the image memory and displayed on the display unit 8.

In the present Specification, the term “image” can broadly indicate both a visual image and data representing a visual image. Furthermore, the term “data” can include raw data, which is ultrasound data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display unit 8 include a LED (Light-Emitting Diode) display, an LCD (Liquid Crystal Display), and an organic EL (Electro-Luminescence) display. The display unit 8 displays an ultrasound image.

The memory 9 is any known data storing medium. In one example, the ultrasound image display system includes a non-transitory storing medium and a transitory storing medium as memories. In addition, the ultrasound image display system may also include a plurality of memories. The non-transitory storing medium is, for example, a non-volatile storing medium such as an HDD (Hard Disk Drive: hard disk drive), a ROM (Read-Only Memory), or the like. The non-transitory storing medium may include a portable storing medium such as a CD (Compact Disk), a DVD (Digital Versatile Disk), or the like. A program executed by the processor 7 is stored in the non-transitory storing medium. The transitory storing medium is a volatile storing medium such as a Random Access Memory (RAM) or the like. The memory 9 does not need to be present on-site, but can be distributed on a cloud connected via a communication network.

The memory 9 stores one or a plurality of instructions that can be executed by the processor 7. The one or plurality of instructions cause the processor 7 to execute various types of operations.

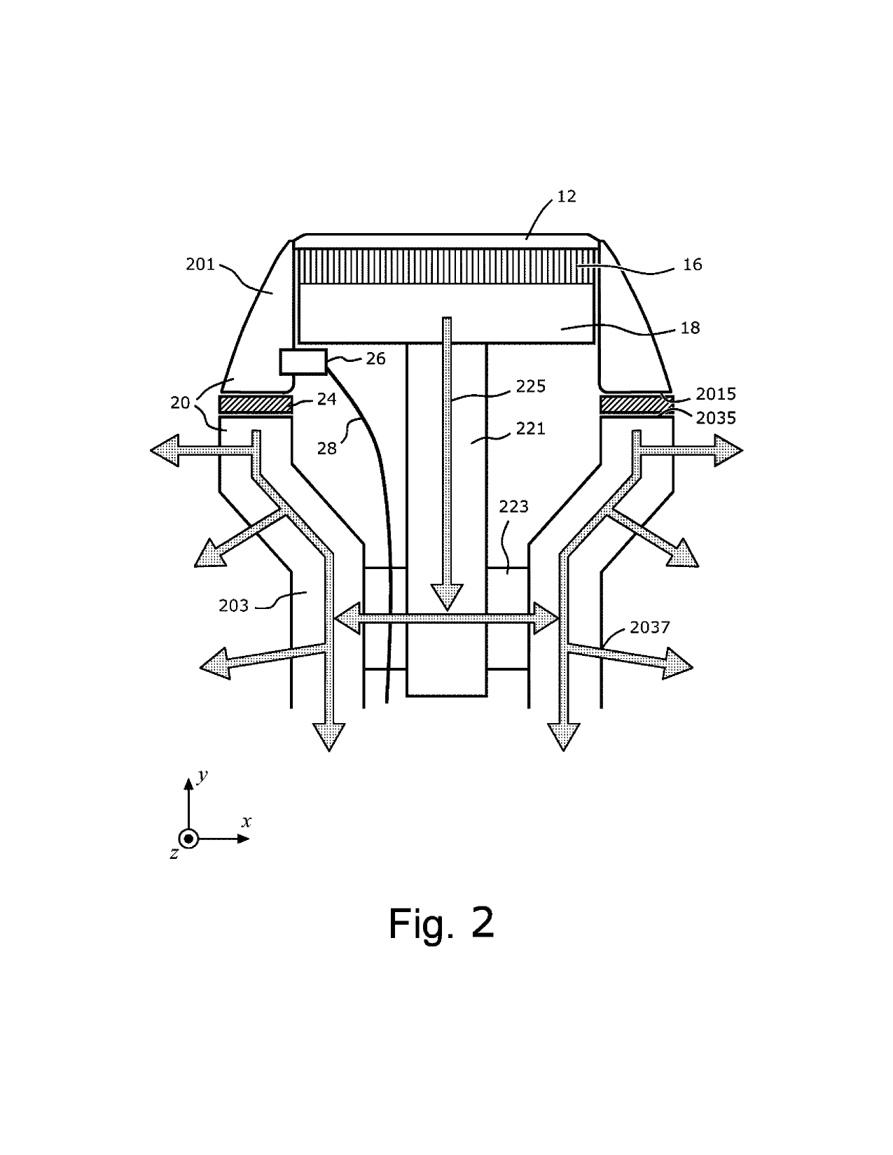

FIG. 2 is a diagram depicting an internal structure of the ultrasonic probe 2 depicted in FIG. 1, and is a front surface view of the inside of the ultrasonic probe 2. In the present embodiment, the ultrasonic probe 2 is a convex-type ultrasonic probe, but the ultrasonic probe may also be an ultrasonic probe for a bronchial endoscope, a transesophageal ultrasonic probe, or another type of ultrasonic probe. The convex-type ultrasonic probe has a longitudinal axis (y-axis) extending vertically on the plane of FIG. 2, an x-axis extending horizontally on the plane of FIG. 2, and a z-axis extending in depthwise on the plane of FIG. 2.

A convex-type ultrasonic probe is generally used for abdominal ultrasound examinations, and the like. The convex-type ultrasonic probe has an outer case 20 with a width in the x-axis direction that is greater than the thickness thereof in the z-axis direction. The cross section orthogonal to the y-axis (cross section along the xz plane) of the outer case 20 of the convex-type ultrasonic probe has a shape close to a rectangle with rounded corners. FIG. 2 depicts the internal structure of the ultrasonic probe 2 with a front portion of the outer case removed to expose an internal structure.

As depicted in FIG. 2, an ultrasonic vibrator 16 is disposed in front of the ultrasonic irradiating device. The ultrasonic vibrator 16 in FIG. 2 corresponds to the vibrating element 2a in FIG. 1. The ultrasonic vibrator 16 may include a crystal element that is susceptible to damage from an external impact. The ultrasonic vibrator 16 generates ultrasonic waves by vibration, and also generates heat as a by-product. The heat-conduction component 22 is thermally connected to the ultrasonic vibrator directly or via another member, and transfers the heat generated in the ultrasonic vibrator toward a rear end direction of the ultrasonic probe 2. In the example of FIG. 2, the heat-conduction component 22 is thermally connected to the ultrasonic vibrator 16 via a signal extraction/thermal connection structure 18. The signal extraction/thermal connection structure 18 is thermally connected to the ultrasonic vibrator 16. In the case of the ultrasonic probe 2 for acquiring an ultrasound image, the temperature of the ultrasonic vibrator 16 rises to about 45° C during use. Contact with metal at 45° C is often too hot for the subject of contact, and is expected to cause unpleasant stress. In the example of FIG. 2, the heat-conduction component 22 includes: a first heat-conduction component 221 extending in the longitudinal axis direction (y-axis direction); and a second heat-conduction component 223 extending in the lateral direction (x-axis direction). The first heat-conduction component 221 and the second heat-conduction component 223 are thermally connected to each other. When connecting the heat-conduction components together, in addition to securing the components together with a fastener, by welding, or the like, the contact area therebetween can be increased by applying a known heat-conducting grease containing metal particles.

The outer case 20 includes: a front portion 201; a rear portion 203; and a heat-insulation component 24 disposed between the front portion 201 and the rear portion 203. The rear portion 203 includes a handle portion for an operator of the ultrasonic probe 2 to hold the ultrasonic probe 2. In this example, the front portion 201 of the outer case 20 is made of metal and surrounds the ultrasonic vibrator 16. The metal material of the front portion 201 of the outer case 20 has higher durability and impact resistance than the resin material that has been used for the outer cases of conventional ultrasonic irradiating devices. This significantly reduces the possibility that the tip end portion of the probe or an internal crystal element will be damaged if the ultrasonic probe 2 is accidentally dropped or hit. The front end of the ultrasonic vibrator 16 is covered with an acoustic lens 12 made of resin. The front portion 201 of the outer case 20 can protect the ultrasonic vibrator 16 and the acoustic lens 12 from external impacts and stresses to a greater extent than conventional outer cases made of resin. The front portion 201 of the outer case 20 can be made of stainless steel, titanium, aluminum alloy, or the like. The first heat-conduction component 221 and the second heat-conduction component 223 can be made of aluminum, an aluminum alloy, copper, a copper alloy, or the like, which have high thermal conductivity. The heat-conduction component 22 may be processed to enhance the thermal conductivity thereof, for example by being coated with copper or the like. An adhesive is applied between the front portion 201 of the metal outer case 20 and the ultrasonic vibrator 16. The front portion 201 of the metal outer case 20 and the ultrasonic vibrator 16 are thermally insulated from each other by the adhesive or a combination of the adhesive and the acoustic lens 12. The adhesive used herein may be, for example, an epoxy resin adhesive or a polyvinyl chloride (PVC) adhesive. When assembling the ultrasonic probe, an adhesive used in other portions may also be an epoxy resin adhesive or a polyvinyl chloride (PVC) adhesive.

In the example of FIG. 2, the front end of the first heat-conduction component 221 is thermally connected to the ultrasonic vibrator 16 via a signal extraction/thermal connection structure 18. The rear end of the first heat-conduction component 221 is thermally connected to a center part of the second heat-conduction component 223. Both ends of the second heat-conduction component 223 are thermally connected to the rear portion 203 of the outer case 20. As described above, the rear portion 203 can include a handle portion for an operator of the ultrasonic probe 2 to hold the ultrasonic probe 2, and in a certain embodiment, the second heat-conduction component 223 is thermally connected to the handle portion of the rear portion 203. As described above, in the case of the ultrasonic probe 2 for acquiring an ultrasound image, the temperature of the ultrasonic vibrator 16 rises to about 45° C during use. Contact with metal at 45° C is often too hot for the subject of contact, and is expected to cause unpleasant stress. However, heat transferred from the heat-conduction component 22 to the handle portion often reduces the temperature to a level that the operator finds comfortable rather than too hot.

The rear portion 203 of the outer case 20, similar to the front portion 201, can be made of stainless steel, titanium, aluminum alloy, or the like. The rear portion 203 of the outer case 20 is thermally connected to the ultrasonic vibrator 16 via the first heat-conduction component 221 and the second heat-conduction component 223, thereby enabling the heat generated by the ultrasonic vibrator 16 to be dissipated from the rear portion 203 of the outer case 20. The plurality of arrows in FIG. 2 indicate the heat-conduction path 225 and the heat dissipation. The rear portion 203 of the outer case 20 may be made of the same metal as the front portion 201, or may be made of a different metal. In another embodiment, the rear portion 203 of the outer case 20 is made of a resin material. In another embodiment, the rear portion 203 of the outer case 20 is made of a combination of a resin material and a metal material. When the rear portion 203 of the outer case 20 is made of a combination of resin material and a metal material, the portion where the operator of the ultrasonic probe 2 is expected to hold the ultrasonic probe 2 can be made of resin, and the other portion can be made of metal. If the rear portion 203 of the outer case 20 is made of resin where the operator of the ultrasonic probe 2 holds the ultrasonic probe 2 and another portion is made of metal, there is an advantage in which heat from the heat- conduction component 22 is prevented from being directly transmitted to the operator, while heat dissipation from the metal portion of the rear portion 203 is promoted. The metal portions of the front portion 201 and rear portion 203 may be coated with various coatings to improve resistance to acid corrosion and to make the portions more pleasant to the touch. When the rear portion 203 of the outer case 20 is made of only metal, the heat dissipation performance can be improved compared to when resin is included. The metal portion of the rear portion 203 of the outer case 20 serves as a heat-dissipating part 2037 that is thermally connected to the heat-conduction component 22.

In the example of FIG. 2, the heat-conduction component 22 includes a first heat-conduction component 221 and a second heat-conduction component 223 combined in an inverted T shape, but the heat-conduction component 22 can also be a single component formed in an inverted T shape. Furthermore, various shapes demarcated by straight lines and curves, such as an inverted Y shape, an inverted V shape, an inverted U shape, an X shape, an O shape, and the like, can be adopted. The heat-conduction component 22 does not need to be flat, but may have a three-dimensional shape, such as a trumpet shape, a bamboo broom shape, or the like, and the heat-conduction component 22 can be connected not only to a side portion of the rear portion 203, but also to a front surface portion or back surface portion of the rear portion 203. This increases the number of heat-dissipation paths and improves the heat-dissipating effect. In a certain embodiment, the heat-conduction component 22 is connected to the rear portion 203 of the outer case 20 at a position other than where an operator is expected to grip the ultrasonic probe assembly. This prevents the heat transmitted from the ultrasonic vibrator 16 from being directly transmitted to the operator's hands.

The heat-insulation component 24 disposed between the front portion 201 and the rear portion 203 of the outer case 20 thermally insulates the front portion 201 from the rear portion 203. The heat-insulation component 24 is formed from a resin having a thermal conductivity of 0.38 W/m·K or less. Specifically, the resin can be made of polyphenylene sulfide, polyvinylidene fluoride, polyamideimide, ethylene tetrafluoroethylene, polycarbonate, polyethylene terephthalate (PET), polybutylene terephthalate (PET), or the like. The resin from which the heat-insulation component 24 is made becomes a part of the outer case 20, and thus is required to have not only low thermal conductivity but also, in principle, high water resistance, chemical resistance, and impact resistance. The ultrasonic probe 2 includes an electronic component connected to the ultrasonic vibrator 16 inside the outer case 20. The electronic component is vulnerable to moisture and/or a chemical that are expected to come into contact with the outer case 20. The heat-insulation component 24 is required to have water resistance and chemical resistance so as not to become a path for the penetration of moisture or chemicals. However, for example, although Bakelite is a water-absorbent material, Bakelite can also be used as the heat-insulation component 24 by applying a waterproofing treatment to the heat-insulation component 24 itself or to the built-in electronic component 2b. The heat-insulation component 24 can be fabricated by at least one of injection molding, extrusion molding, blow molding, vacuum molding, compression molding, 3D printing, and cutting and processing.

The heat-insulation component 24 may be a solid member formed from the abovementioned resin. In another embodiment, the heat-insulation component 24 may be a hollow member formed from the abovementioned resin. An internal cavity of the hollow member can be filled with air or an inert gas. The internal cavity of the hollow member can be reinforced with a reinforcing member having a honeycomb structure, for example, to prevent deformation due to stress generated during use of the ultrasonic probe 2. In another embodiment, the heat-insulation component 24 can be a vacuum insulation panel (VIP). The vacuum insulation panel (VIP) can be made by placing an insulating core material, such as glass fiber, urethane foam, or the like, in an outer bag made of a composite film with gas barrier properties and creating a vacuum inside. Vacuum insulation panels (VIPs) are used as insulation panels for refrigerators, and the like.

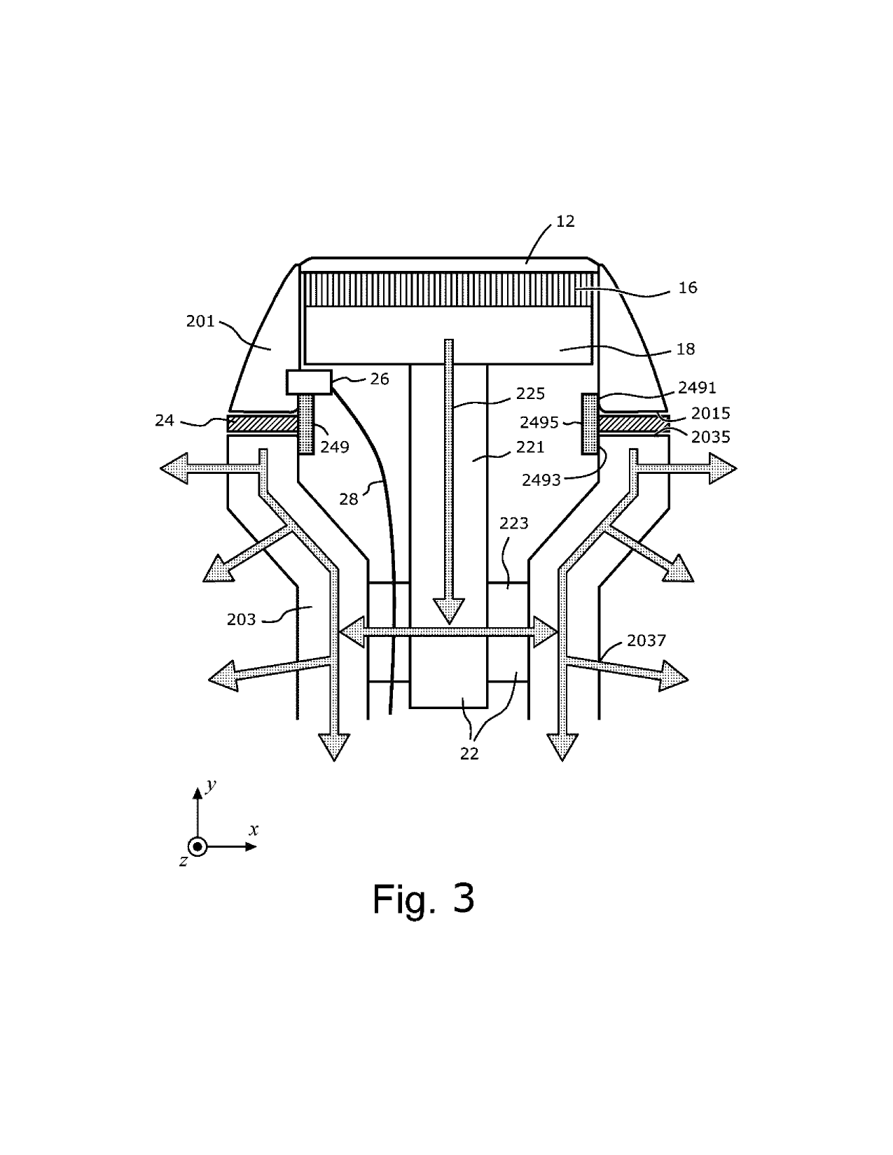

The heat-insulation component 24 in FIG. 2 can be replaced with, for example, the form depicted in FIG. 3. The heat-insulation component 24 depicted in FIG. 3 includes a cylindrical member 249. The cylindrical member 249 includes: an inner surface 2495; a first outer surface 2491 facing an inner surface of the front portion 201 of the outer case 20; and a second outer surface 2493 facing an inner surface of the rear portion 203 of the outer case 20. The first surface 241 and the second surface 243 of the heat-insulation component 24 extend from the cylindrical member 249 to the outer surface of the outer case 20. The flange member of the heat-insulation component 24 is demarcated by a first surface 241 and a second surface 243. The cylindrical member 249 and flange member of the heat-insulation component 24 are fabricated as a single integrally formed member. In another embodiment, the cylindrical member 249 and flange member of the heat-insulation component 24 are fabricated as separate components and adhered together. Both can be made of different materials or the same material. The first outer surface 2491 and the second outer surface 2493 of the cylindrical member 249 may be joined, by an adhesive, to the inner surfaces of the front portion 201 and the rear portion 203, respectively. When the first outer surface 2491 and the second outer surface 2493 of the cylindrical member 249 are joined, by an adhesive, to the inner surface of the front portion 201 and the inner surface of the rear portion 203, respectively, the strength against a shear force acting in a direction orthogonal to the y-axis can be increased.

As depicted in FIGS. 2 and 3, a heater 26 can optionally be disposed on an inner wall of the front portion 201 of the outer case 20. The heater 26 is thermally connected to the front portion 201 and is capable of increasing the temperature of the front portion 201. The heater 26 can include an electric heating wire that extends around an entire periphery of the inner wall of the front portion 201 of the outer case 20. The electric heating wire can also be replaced with an electric heating film that generates surface heat. The heater 26 is configured to generate heat at 30° C to 70° C, more preferably 40° C to 60° C, and even more preferably 45° C to 55° C. The heaters 26 can be arranged to cover all positions that may be contacted by a patient serving as a subject of contact. The heater 26 receives electrical power from a power line 28 that transfers power from a cable 30 (to be described later) to the heater 26 and generates heat.

The heater 26 can be turned ON/OFF automatically in accordance with a temperature sensor for detecting the temperature of the front portion 201 of the outer case 20. The temperature sensor may be a mechanical thermostatic switch containing a bimetal. Furthermore, the temperature sensor can be an electrical sensor using a thermistor or thermocouple.

In another embodiment, the heater 26 can be turned ON/OFF by a manual switch disposed at the rear portion 203 of the outer case 20 of the ultrasonic probe 2. The ON/OFF control of the heater 26 can also be performed by an operator input via the user interface 10 (FIG. 1). The operator of the ultrasonic probe 2 can check the temperature by touching the front portion 201 of the outer case 20 and can turn the heater 26 ON/OFF by the manual switch. In a certain embodiment, the front portion 201 of the outer case 20 includes a portion to which a thermo-pigment or paint that changes color with temperature is applied. The operator of the ultrasonic probe 2 can check the temperature of the front portion 201 by visually checking the portion to which the thermo-pigment or paint that changes color with temperature is applied, and can turn the heater 26 ON/OFF by a manual switch.

In the ultrasonic probe 2 having the front portion 201 of the metal outer case 20, the heat-insulation component 24 thermally separates the front portion 201 that comes into contact with a patient from the rear portion 203 that contributes to heat dissipation. In addition thereto, the heater 26 adjusts the temperature of the front portion 201 to an appropriate level before use of the ultrasonic probe 2, thereby reducing the risk of causing thermal discomfort to the patient while maintaining heat dissipation performance.

Returning to FIG. 1, the ultrasound diagnostic system 1 includes a probe holder 11 that internally accommodates at least a tip-end part of the probe 2. The probe holder 11 can be disposed at a position that is easy for an operator to operate, such as beside a keyboard of the ultrasound diagnostic system 1, on a headboard of an examination bed on which a patient lies, or the like. The probe holder 11 is provided with a heater 11a. The heater 11a of the probe holder 11 is located outside the probe 2 and can therefore be called an external heater 11a. When the ultrasound diagnostic system 1 is in operation, the probe holder 11 can activate the heater 11a upon detecting that the probe 2 is inserted into the probe holder 11. Furthermore, when the probe holder 11 detects that the probe 2 has been removed from the probe holder 11, the heater 11a can be turned OFF. A state in which the probe 2 is held by the probe holder 11 can be detected by a known detection technology, such as a contact sensor, a magnetic sensor, an optical sensor, or the like. The operating state of the heater 11a of the probe holder 11 can be indicated to the operator by an indicator 11b provided on the probe holder 11. The indicator 11b of the probe holder 11 can also display whether or not the probe 2 is correctly set in the probe holder 11. The probe holder 11 can be attached to a bottle holder that holds a bottle of ultrasound diagnostic gel. The bottle holder can include a heater for heating the ultrasound diagnostic gel to a temperature close to body temperature. When the probe holder 11 is provided next to the bottle holder, the probe 2 held in the probe holder 11 can be heated by using the heater of the bottle holder. When the tip-end part of the probe 2 is heated by the probe holder 11, the heater 26 and the power line 28 internally provided in the outer case 20 depicted in FIG. 2 can be made unnecessary components.

The description will continue with reference to FIGS. 1 and 2. Whether the front portion 201 of the outer case 20 of the probe 2 is heated by the probe holder 11 or by a heater 26 internally provided in the outer case 20, a smaller front portion 201 volume of the outer case 20 allows the temperature of the front portion 201 to be raised to the desired temperature more rapidly. On the other hand, if the volume of the front portion 201 of the outer case 20 is too small, a region for providing the heater 26 may not be ensured, or the ultrasonic vibrator 16 may not be sufficiently protected. In a preferred embodiment, the heat-insulation component 24 is disposed at a position where the volume of the front portion 201 is 5-30% of the total volume of the front portion 201 and the rear portion 203. More preferably, the heat-insulation component 24 is disposed at a position where the volume of the front portion 201 is 8-20% of the total volume of the front portion 201 and the rear portion 203. When an electric heating wire is used for the heater 26, the electric heating wire has the advantage in which a region that is wide in the longitudinal axis direction is not required.

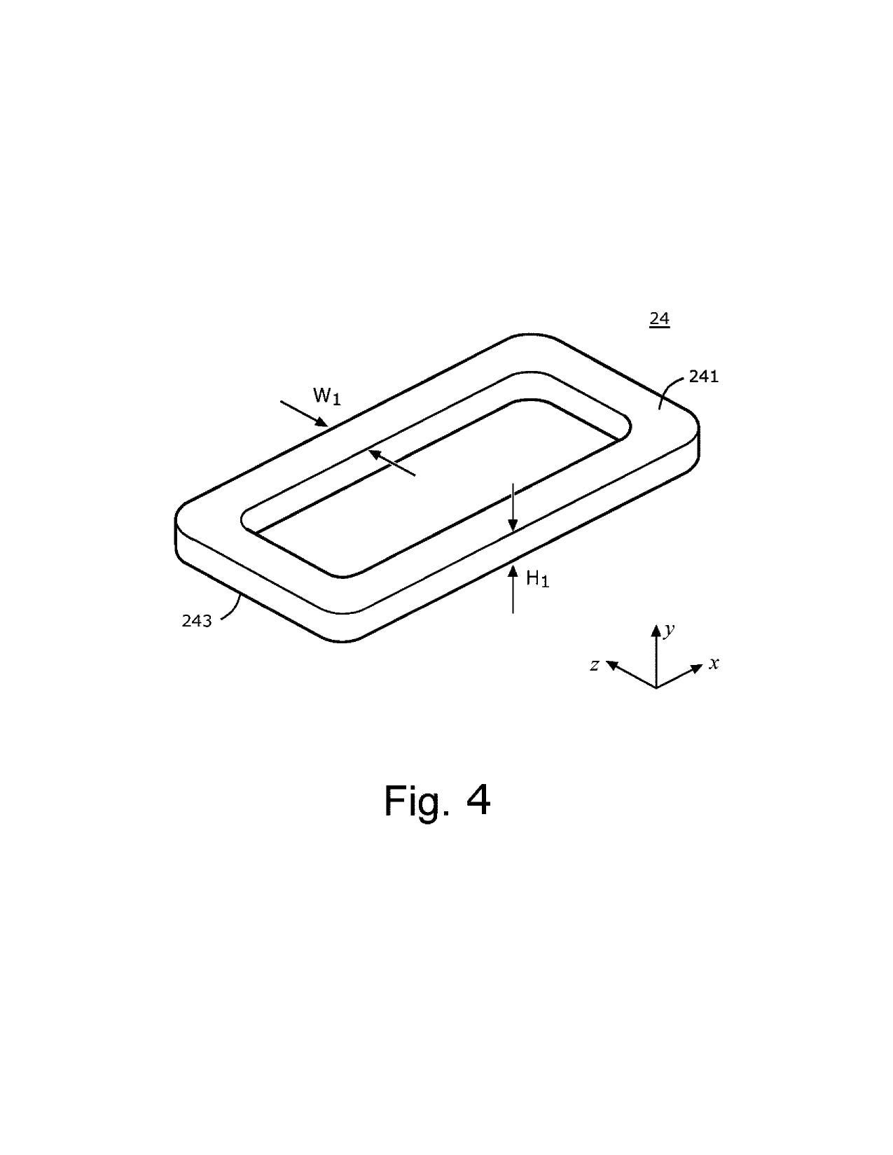

FIG. 4 is an enlarged view of the heat-insulation component 24 depicted in FIG. 2. The heat-insulation component 24 has: a first surface 241 adhered to an end surface 2015 of the front portion 201 of the outer case 20; and a second surface 243 adhered to an end surface 2035 of the rear portion 203 of the outer case 20. Therefore, the completed outer case will have: a first adhesive layer between the front portion 201 and the first surface 241; and a second adhesive layer between the rear portion 203 and the second surface 243. In the example of FIG. 3, the first surface 241 and the second surface 243 are parallel to a plane orthogonal to the longitudinal axis (y-axis) of the ultrasonic probe 2. In another embodiment, the first surface 241 and the second surface 243 are not parallel to a plane orthogonal to the longitudinal axis (y-axis) of the ultrasonic probe 2, but extend in a direction inclined relative to such a plane. When the first surface 241 and/or the second surface 243 extend in a direction inclined with respect to the plane perpendicular to the longitudinal axis (y-axis), the strength against a shear force acting in a direction orthogonal to the y-axis can be increased.

The thickness H1 of the heat-insulation component 24 along the longitudinal axis (y-axis) must be thick enough to provide sufficient heat resistance between the front portion 201 and the rear portion 203. The thickness H1 is 1 mm or more, and may be between 1 mm and 10 mm, and preferably between 3 mm and 7 mm.

The width W1 of the heat-insulation component 24 can be the same as the width of the end surfaces 2015 and 2035 of the outer case 20. An inner surface of the heat-insulation component 24 can be formed to match the inner surfaces of the front portion 201 and the rear portion 203, and an outer surface of the heat-insulation component 24 can be formed to match the outer surfaces of the front portion 201 and the rear portion 203. In another embodiment, the inner surface of the heat-insulation component 24 may not match the inner surfaces of the front portion 201 and the rear portion 203, and the inner surface of the heat-insulation component 24 may be formed to be on an inner side from the inner surfaces of the front portion 201 and the rear portion 203. Furthermore, the outer surface of the heat-insulation component 24 does not coincide with the outer surfaces of the front portion 201 and the rear portion 203, and the outer surface of the heat-insulation component 24 can be formed so as to be on the outer side from the outer surfaces of the front portion 201 and the rear portion 203.

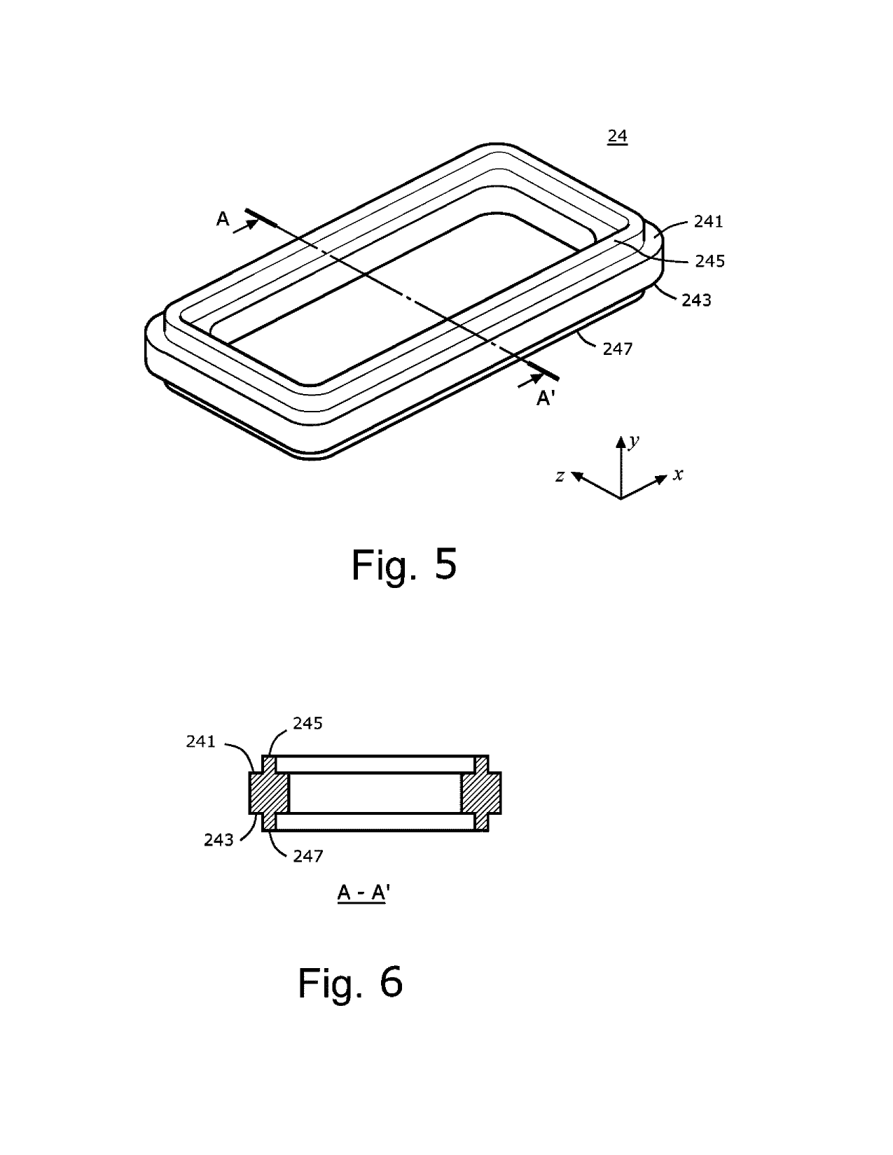

FIG. 5 is an enlarged view of an alternative example of the heat-insulation component 24 depicted in FIG. 2. Similar to the heat-insulation component 24 of FIG. 4, the heat-insulation component 24 has: a first surface 241 adhered to an end surface 2015 of the front portion 201 of the outer case 20; and a second surface 243 adhered to an end surface 2035 of the rear portion 203 of the outer case 20. In the example of FIG. 5, in addition thereto, the first surface 241 has a first protrusion 245 protruding toward the front portion 201, and the second surface 243 has a second protrusion 247 protruding toward the rear portion 203. FIG. 6 is a cross-sectional view depicting a state in which a part of the heat-insulation component 24 depicted in FIG. 5 is cut. In this example, the front portion 201 has a groove for receiving a first protrusion 245 and the rear portion 203 also has a groove for receiving a second protrusion 247. If the front portion 201 has a groove for receiving a first protrusion 245 and the rear portion 203 has a groove for receiving the second protrusion 247, the strength against a shear force acting in a direction orthogonal to the y-axis can be increased. In the examples of FIGS. 5 and 6, the first protrusion 245 and the second protrusion 247 both have a shape with a substantially rectangular cross section, but can also be changed to another shape including a triangular shape. A dovetail joint can be used to link the first protrusion 245 and the second protrusion 247 to corresponding grooves. When the first protrusion 245 and the second protrusion 247 are in a flexible state, the protrusions can be inserted into corresponding grooves. The dovetail joint can be used to increase resistance to tensile stress along the y-axis. In the example of FIG. 5, the first protrusion 245 and the second protrusions 247 are formed around an entire periphery of the heat-insulation component 24. However, the protrusions do not need to be formed around the entire periphery, and the first protrusion 245 and the second protrusion 247 can be formed in limited positions, for example, only at the four corners, only at center parts of the four sides, or the like.

In the description above, in many cases, the ultrasonic irradiating device has been described as an example of the ultrasonic probe 2 for capturing an ultrasound image. The ultrasonic probe 2 for capturing an ultrasound image generally emits a frequency of 2 to 18 MHz at an output intensity of 0.1 to 720 mW/cm². Note that as is clear to a person of ordinary skill in the art, higher frequencies provide more detailed images but also have reduced penetration through tissue. Lower frequencies penetrate deeper but provide lower resolution.

In contrast, when the ultrasonic irradiating device is an ultrasonic treatment device for destroying a tumor in vivo, the ultrasonic vibrator 16 generally irradiates ultrasonic waves with a frequency of 0.8 to 3 MHz at an output intensity of 100 to 10,000 W/cm². The irradiated ultrasonic waves are focused onto a specific site, concentrating high energy to heat or destroy tissue at that site. If the target is a tumor cell, the tumor cell can be destroyed with thermal energy. Furthermore, when the ultrasonic irradiating device is a nerve stimulating device for stimulating a nerve in vivo and causing the release of a prescribed neurotransmitter, the ultrasonic vibrator 16 generally irradiates ultrasonic waves with a frequency of 0.2 to 1 MHz at an output intensity of 1 to 100 mW/cm². The nerve stimulating device is used in the field of diabetes treatment and the like, for example. In either case, a temperature higher than that of the ultrasonic vibrator 16 of the ultrasonic probe 2 for capturing an ultrasound image is expected to be generated. Therefore, in the case of the ultrasonic treatment device and nerve stimulating device, the thickness of the heat-insulation component 24 is required to be thicker than that of the ultrasonic probe 2, and to be thick enough to provide sufficient heat resistance between the front portion 201 and the rear portion 203.

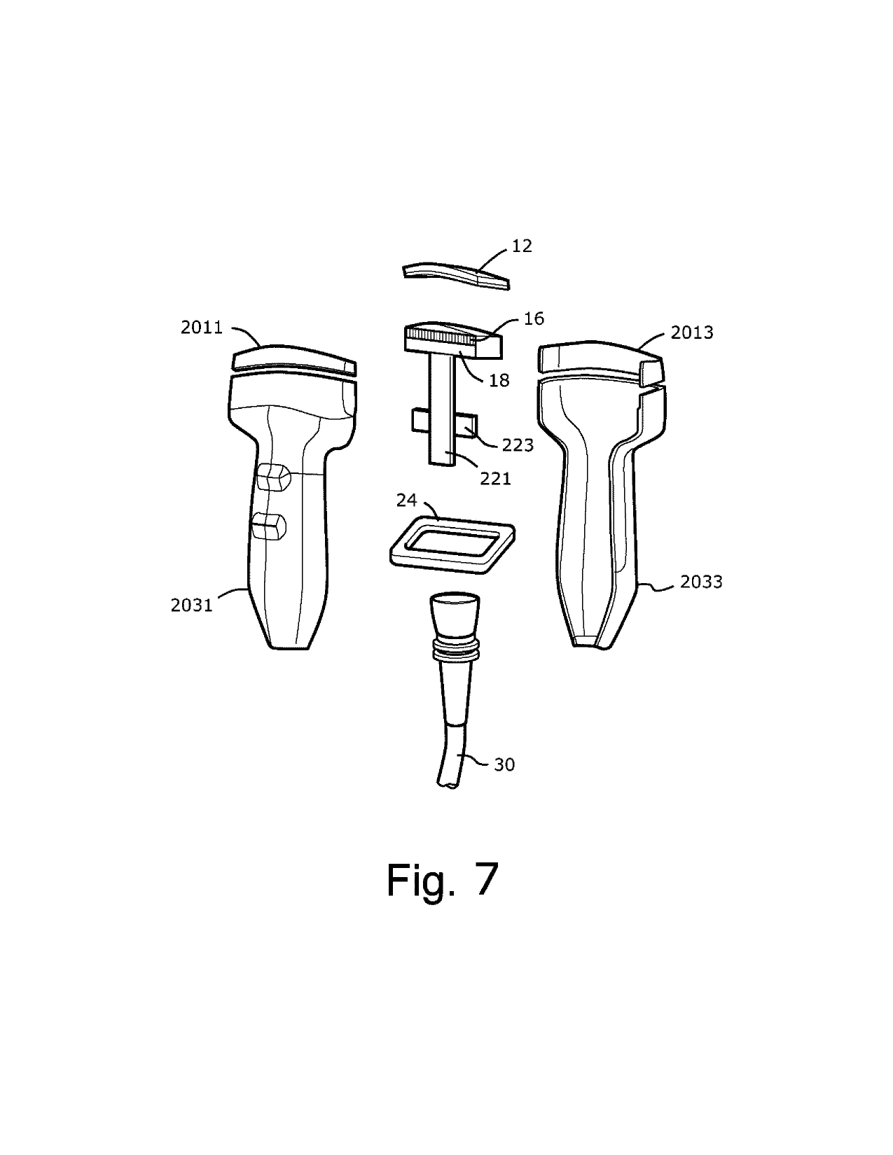

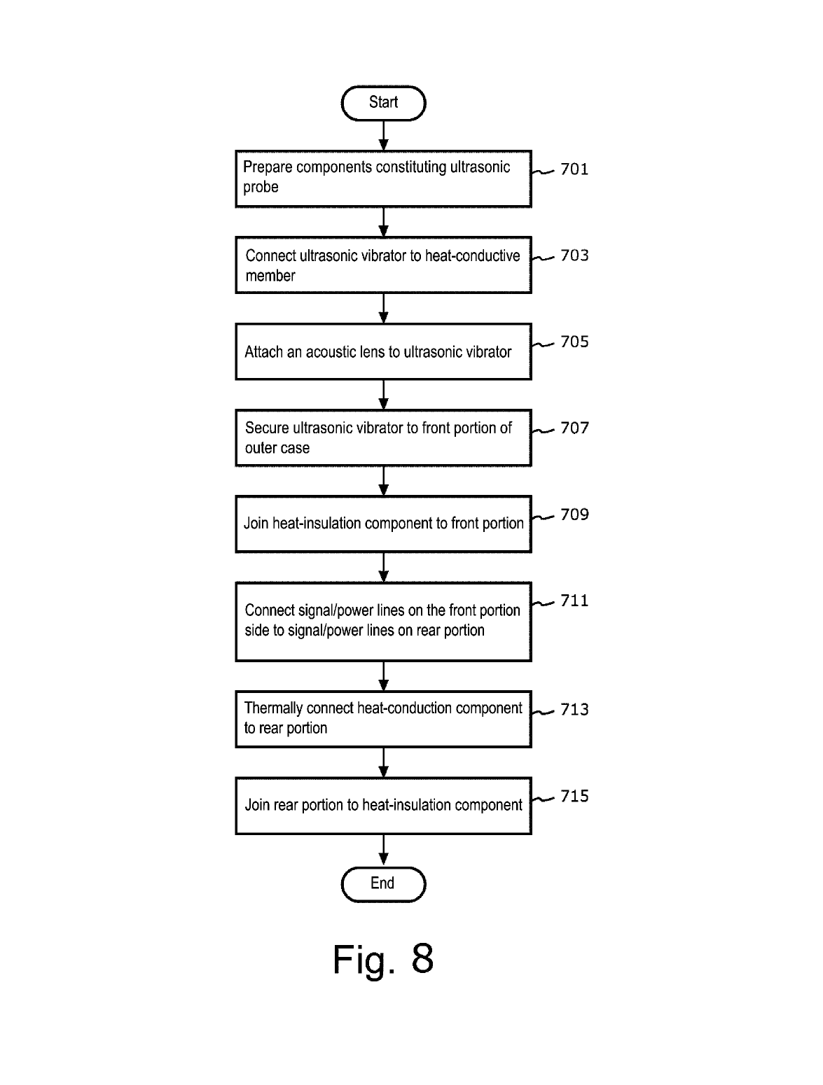

FIG. 7 is an exploded perspective view depicting an internal structure of the ultrasonic probe 2. FIG. 8 is a flowchart depicting a method for manufacturing the ultrasonic probe 2. The method for manufacturing the ultrasonic probe 2 will be described with reference to FIGS. 7 and 8. Herein, a description will also be given taking as an example that the ultrasonic irradiating device is the ultrasonic probe 2 for capturing an ultrasound image, but the same can be done for the ultrasonic treatment device, nerve stimulating device, or the like. It should be understood that the described steps and the order thereof are provided for illustrative purposes only, and that in reality, specific actions may be executed in a different order or in parallel with one another. In actuality, the steps described and the order thereof are provided merely for illustrative purposes, to provide real-world implementation examples, and should not be considered limiting.

In step 701, components constituting the ultrasonic probe 2 are prepared. Step 701 involves a step for fabricating a heat-insulation component. The heat-insulation component can be fabricated using a known technique, such as injection molding, extrusion molding, blow molding, vacuum molding, compression molding, 3D printing, cutting and processing, or the like. In step 703, the ultrasonic vibrator 16 is connected to the front-end part of the first heat-conduction component 221. The ultrasonic vibrator 16 can be connected to the first heat-conduction component 221 directly or via another component, such as the signal extraction/thermal connection structure 18 or the like. Components can be connected using welding, brazing, a screw or other fastener, an adhesive, or the like.

In step 705, the acoustic lens 12 is attached to the ultrasonic vibrator 16. In a certain embodiment, an adhesive is used to attach the acoustic lens 12 to the ultrasonic vibrator 16. This step may not be necessary if the ultrasonic vibrator is prefabricated to include the function of an acoustic lens, or the like. In step 707, the ultrasonic vibrator 16 (with the acoustic lens 12 attached) is secured to the front portion 201 of the outer case 20. The ultrasonic vibrator 16 can be secured to the front portion 201 of the outer case 20 by adhering the acoustic lens 12 to the front portion 201 using an adhesive and/or by adhering the ultrasonic vibrator 16 to the front portion 201 using an adhesive. As depicted in FIG. 7, the front portion 201 of the outer case 20 can also be assembled by dividing the front portion 201 into a front surface portion 2011 and a back surface portion 2013. The front surface portion 2011 and the back surface portion 2013 of the front portion 201 can be connected using welding, brazing, a screw or other fastener, an adhesive, or the like.

In step 709, the heat-insulation component 24 is joined to the front portion 201. The joining can be performed using an adhesive. In step 711, a signal line and power line on the front portion 201 side are connected to a signal line and power line on the rear portion 203 side. In step 713, the heat-conduction component 22 and the rear portion 203 of the outer case 20 are thermally connected. As depicted in FIG. 7, when the rear portion 203 is divided into a front surface portion 2031 and a back surface portion 2033, the heat-conduction component 22 and the rear portion 203 can be connected using welding, brazing, a screw or other fastener, an adhesive, or the like. If the rear portion 203 is divided into the front surface portion 2031 and the back surface portion 2033, after all the steps in FIG. 8 are completed, the front surface portion 2031 and the back surface portion 2033 can be finally joined and adhered to the heat-insulation component 24. Regardless of whether the rear portion 203 is divided into the front surface portion 2031 and the back surface portion 2033, the heat-conduction component 22 and the rear portion 203 can be connected, for example, by inserting the heat-conduction component 22 into a slot that is provided in the rear portion 203 and that has heat-conducting grease provided therein. Furthermore, by making the heat-conduction component 22 a linking mechanism that can expand and contract in the y-axis direction, similar to a train pantograph or a toy magic hand, the heat-conduction component 22 can be joined to each of the separated front portion 201 and rear portion 203, which are then brought close to each other while maintaining the heat-conduction path. Thus, the two can be connected via the heat-insulation component 24. In step 715, the heat-insulation component 24 is joined to the rear portion 203. The joining can be performed using an adhesive.

Note that the invention is not limited to the present embodiment, and various modifications are possible without departing from the gist of the invention. The present specification has been described using embodiments to disclose the present subject matter, including a preferred embodiment, and also to enable a person of ordinary skill in the art to implement the subject matter, including manufacturing and using of any device or system, and to execute an incorporated method. The patentable scope of the subject matter is defined by the claims, and may include another example conceived of by a person of ordinary skill in the art. Such another embodiment is intended to be within the scope of the claims if it has structural elements that do not differ from the literal language of the claims, or if it includes equivalent structural elements that do not substantially differ from the literal language of the claims.

### DESCRIPTION OF CODES

1: Ultrasound diagnostic system

2: Ultrasonic probe

2a: Vibrating element

2b: Electronic component

3: Transmission beamformer

4: Transmitter

5: Receiver

6: Reception beamformer

7: Processor

8: Display unit

9: Memory

10: User interface

11: Probe holder

11a: External heater

11b: Indicator

12: Acoustic lens

16: Ultrasonic vibrator

18: Signal extraction/thermal connection structure

20: Outer case

201: Front portion

2011: Front surface portion

2013: Back surface portion

2015: End surface

203: Rear portion

2031: Front surface portion

2033: Back surface portion

2035: End surface

2037: Heat-dissipating part

22: Heat-conduction component

221: First heat-conduction component

223: Second heat-conduction component

225: Heat-conduction path

24: Heat-insulation component

241: First surface

243: Second surface

245: First protrusion

247: Second protrusion

249: Cylindrical member

2491: First outer surface

2493: Second outer surface

2495: Inner surface

26: Heater

28: Power line

30: Cable

1. An ultrasonic irradiating device, comprising:
an ultrasonic vibrator disposed in front of the ultrasonic irradiating device;
an outer case surrounding the ultrasonic vibrator; and
a heat-conduction component thermally connected to the ultrasonic vibrator and transferring heat generated by the ultrasonic vibrator, wherein
the outer case includes a front portion made of metal, a rear portion, and a heat-insulation component disposed between the front portion and the rear portion, and the rear portion of the outer case and the heat-conduction component are thermally connected.

2. The ultrasonic irradiating device according to claim 1, further comprising a heater thermally connected to the front portion of the outer case.

3. The ultrasonic irradiating device according to claim 2, wherein the rear portion of the outer case includes a metal material.

4. The ultrasonic irradiating device according to claim 2, wherein the heat-insulation component is formed from a resin having a thermal conductivity of 0.38 W/m·K or less.

5. The ultrasonic irradiating device according to claim 1, wherein the heat-insulation component has a first surface formed to contact an end surface of the front portion and a second surface formed to contact the end surface of the rear portion; a space between the first surface and the second surface has a thickness that can ensure heat insulation; and the first surface and/or the second surface extends in a plane orthogonal to the longitudinal axis of the ultrasonic irradiating device or in a direction inclined relative to the plane.

6. The ultrasonic irradiating device according to claim 5, wherein
the heat-insulation component includes a cylindrical member;
the cylindrical member includes a first outer surface facing an inner surface of the front portion of the outer case and a second outer surface facing an inner surface of the rear portion of the outer case; and
the first surface and the second surface extend from the cylindrical member to an outer surface of the outer case.

7. The ultrasonic irradiating device according to claim 1, wherein the thickness of the space between the first surface and the second surface is hollow or solid.

8. The ultrasonic irradiating device according to claim 1, wherein the outer case includes: a first adhesive layer between the front portion of the outer case and the heat-insulation component; and a second adhesive layer between the rear portion of the outer case and the heat-insulation component.

9. The ultrasonic irradiating device according to claim 1, wherein the rear portion of the outer case includes a handle portion for an operator of the ultrasonic irradiating device to hold the ultrasonic irradiating device;
the heat-conduction component includes an axial portion extending along the longitudinal axis of the ultrasonic irradiating device and a lateral portion extending in a direction transverse to the longitudinal axis; and
the lateral portion of the heat-conduction component thermally connects the axial portion and the handle portion of the outer case.

10. The ultrasonic irradiating device according to claim 1, further comprising an acoustic lens surrounding an ultrasound irradiation surface of the ultrasonic vibrator.

11. The ultrasonic irradiating device according to claim 2, further comprising:
a temperature sensor for detecting the temperature of the front portion of the outer case; and
a heater control unit for operating the heater in accordance with the temperature detected by the temperature sensor.

12. The ultrasonic irradiating device according to claim 2, wherein the heater includes an electric heating wire extending around the entire periphery of an inner wall of the front portion of the outer case.

13. The ultrasonic irradiating device according to claim 2, wherein the rear portion of the outer case includes a heat-dissipating part thermally connected to the heat-conduction component.

14. The ultrasonic irradiating device according to claim 1, further comprising an electronic component connected to the ultrasonic vibrator, wherein
the electronic component is vulnerable to moisture and/or a chemical that are expected to come into contact with the outer case.

15. The ultrasonic irradiating device according to claim 1, wherein the heat-insulation component has a thickness in a longitudinal direction of the ultrasonic irradiating device that separates the front and rear portions by a distance of 1 mm or more.

16. The ultrasonic irradiating device according to any one of claim 1, wherein the ultrasonic irradiating device is an ultrasonic probe for irradiating ultrasonic waves with a frequency of 2 to 18 MHz at an output intensity of 0.1 to 720 mW/cm² to capture an ultrasound image.

17. The ultrasonic irradiating device according to any one of claim 1, wherein the ultrasonic irradiating device
irradiates ultrasonic waves with a frequency of 0.8 to 3 MHz at an output intensity of 100 to 10,000 W/cm² to destroy tumors in vivo,
or
irradiates ultrasonic waves with a frequency of 0.2 to 1 MHz at an output intensity of 1 to 100 mW/cm² to stimulate a nerve in vivo and release a prescribed neurotransmitter.

18. An ultrasound diagnostic system, comprising:
the ultrasonic irradiating device according to claim 16,
a probe holder for accommodating a tip-end part of the ultrasonic probe, the probe holder including a heater for heating the tip-end part of the ultrasonic probe;
a processor for processing an echo signal received from the ultrasonic probe using an image generation program to generate an ultrasound image; and
a non-transitory storing medium for storing the image generation program.

19. A heat-insulation component for an outer case of an ultrasonic irradiating device, wherein
the ultrasonic irradiating device includes:
an ultrasonic vibrator disposed in front of the ultrasonic irradiating device;
the outer case surrounding the ultrasonic vibrator; and
a heat-conduction component thermally connected to the ultrasonic vibrator and transferring heat generated by the ultrasonic vibrator, and
the outer case includes a front portion made of metal, a rear portion, and the heat-conduction component disposed between the front portion and the rear portion, and the rear portion of the outer case and the heat-conduction component are thermally connected.
